(19) United States
(12) Patent Application Publication
(10) Pub. No.: US 2009/0030436 A1
(43) Pub. Date: Jan. 29, 2009

(54) Hydraulic acuation for microsurgical instruments

(76) Inventor: Steven T. Charles, Memphis, TN (US)

Correspondence Address: ALCON, IP LEGAL, TB4-8, 6201 SOUTH FREEWAY, FORT WORTH, TX, 76134, US

(21) Appl. No.: 12/119,732

(22) Filed: May 13, 2008

Related U.S. Application Data
(60) Provisional application No. 60/952,426, filed on Jul. 27, 2007.

(51) Int. Cl. A61B 17/50

(52) U.S. Cl. 606/161

(57) ABSTRACT

A microsurgical system capable of hydraulic actuation of microsurgical instruments. Such a system will provide greater force/mass and force/volume ratios, allow for better open loop control, and provide force to overcome tissue resistance.

This application claims the priority of U.S. Provisional Application No. 60/952,426 filed Jul. 27, 2007.

### FIELD OF THE INVENTION

The present invention generally pertains to microsurgical systems. More particularly, but not by way of limitation, the present invention pertains to a microsurgical system capable of providing hydraulic actuation to microsurgical instruments.

### DESCRIPTION OF THE RELATED ART

Many microsurgical procedures require precision cutting and/or removal of various body tissues. For example, certain ophthalmic surgical procedures require the cutting and/or removal of the vitreous humor, a transparent jelly-like material that fills the posterior segment of the eye. The vitreous humor, or vitreous, is composed of numerous microscopic fibers that are often attached to the retina. Therefore, cutting and removal of the vitreous must be done with great care to avoid traction on the retina, the separation of the retina from the choroid, a retinal tear, or, in the worst case, cutting and removal of the retina itself.

The use of microsurgical cutting instruments (i.e. vitrectomy probes, powered scissors, or powered forceps) in posterior segment ophthalmic surgery is well known. Such instruments are actuated with pneumatic pressure or electric motors and are typically inserted via an incision in the sclera near the pars plana. The surgeon may also insert other microsurgical instruments such as a fiber optic illuminator, an infusion cannula, or an aspiration probe during the posterior segment surgery. The surgeon performs the procedure while viewing the eye under a microscope.

In such conventional microsurgical instruments, the use of compressible gasses results in a loss of mechanical actuation force. This reduces the precision of open loop control, and causes difficulty overcoming static or tissue resistance.

Therefore, a need exists for improved devices for actuating microsurgical instruments. Such devices would demonstrate more precise open loop control, as well as force to mass and force to volume ratios that far exceed the mechanical capabilities of pneumatic or electrically actuated devices.

### SUMMARY OF THE INVENTION

In a preferred embodiment, the present invention comprises a microsurgical system capable of providing hydraulic actuation of a microsurgical instrument. The microsurgical system has a microsurgical instrument having an internal hydraulic actuator, a computer, a storage reservoir containing a non-compressible hydraulic fluid, a tube fluidly coupling the reservoir and the actuator of the instrument, and a solenoid valve located along the tube.

### BRIEF DESCRIPTION OF THE DRAWINGS

For a more complete understanding of the present invention, and for further objects and advantages thereof, reference is made to the following description taken in conjunction with the accompanying drawings, in which:

FIG. 1 is a schematic view of a microsurgical system of the present invention.

FIG. 2 is an enlarged cross sectional view of a surgical instrument of the microsurgical system of the present invention.

FIG. 3 is a schematic view of a proportional controller of the microsurgical system of the present invention.

FIG. 4 is a schematic view of a second embodiment of a microsurgical system of the present invention.

### DETAILED DESCRIPTION OF THE PREFERRED EMBODIMENTS

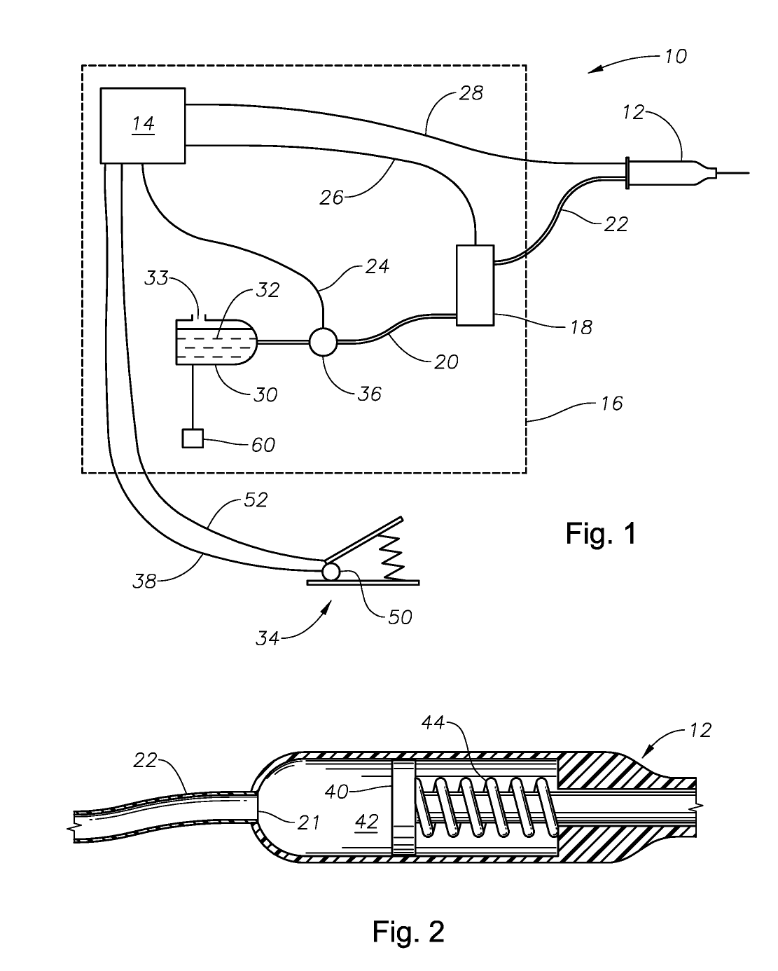

The preferred embodiments of the present invention and their advantages are best understood by referring to FIGS. 1-4 of the drawings, like numerals being used for like and corresponding parts of the various drawings.

FIG. 1 illustrates that microsurgical system 10 comprises microsurgical instrument 12, computer or microprocessor 14, surgical console 16, proportional solenoid valve 18, and user controller 34. Microsurgical instrument 12 is fluidly coupled to valve 18 via tube 22, and is electrically coupled to computer 14 via interface 28. Microsurgical instrument 12 may be any microsurgical instrument having mechanically driven components such as a vitreous cutter, powered proportional scissors, or powered proportional forceps, but is most preferably powered proportional scissors. As best shown in FIG. 2, microsurgical instrument 12 has hydraulic actuator 40 disposed therein. Hydraulic actuator 40 may be any mechanism appropriate for transmitting mechanical force such as a diaphragm, bellows, piston, or bourdon actuator, but is most preferably a diaphragm or bellows. Actuator 40 is mechanically coupled, at the distal end, to a movable cutting or gripping member (not shown), and is disposed within cylinder 42 which is fluidly coupled, at its proximal end, to tube 22 via port 21. Spring 44 applies a restoring force on actuator 40.

Computer 14 is preferably integrated within surgical console 16, but may alternatively be a stand alone unit. Surgical console 16 has fluid reservoir 30 disposed therein. Reservoir 30 contains hydraulic fluid 32, and is fluidly coupled to valve 18 via tube 20. Fluid 32 is preferably a non-compressible hydraulic fluid such as BSS® irrigating solution available from Alcon Laboratories, Inc. of Fort Worth, Tex.; saline solution; or deionized water, and is most preferably sterile saline solution. Fluid 32 may be added to reservoir 30 at the time of equipment manufacture, but is most preferably added by operating room personnel before a surgical procedure via port 33. Reservoir 30 is also fluidly coupled to source of pressure 60. Pressure transducer 36 is fluidly coupled to tube 20 between reservoir 30 and valve 18. Pressure transducer 36 is electrically coupled to computer 14 via interface 24.

Valve 18 is most preferably a proportional solenoid-actuated valve. Valve 18 is electrically coupled to computer 14 via interface 26. Valve 18 is most preferably a bias-closed type valve, such that when no electrical current is applied, valve 18 is closed. Conversely, when current is applied, valve 18 opens.

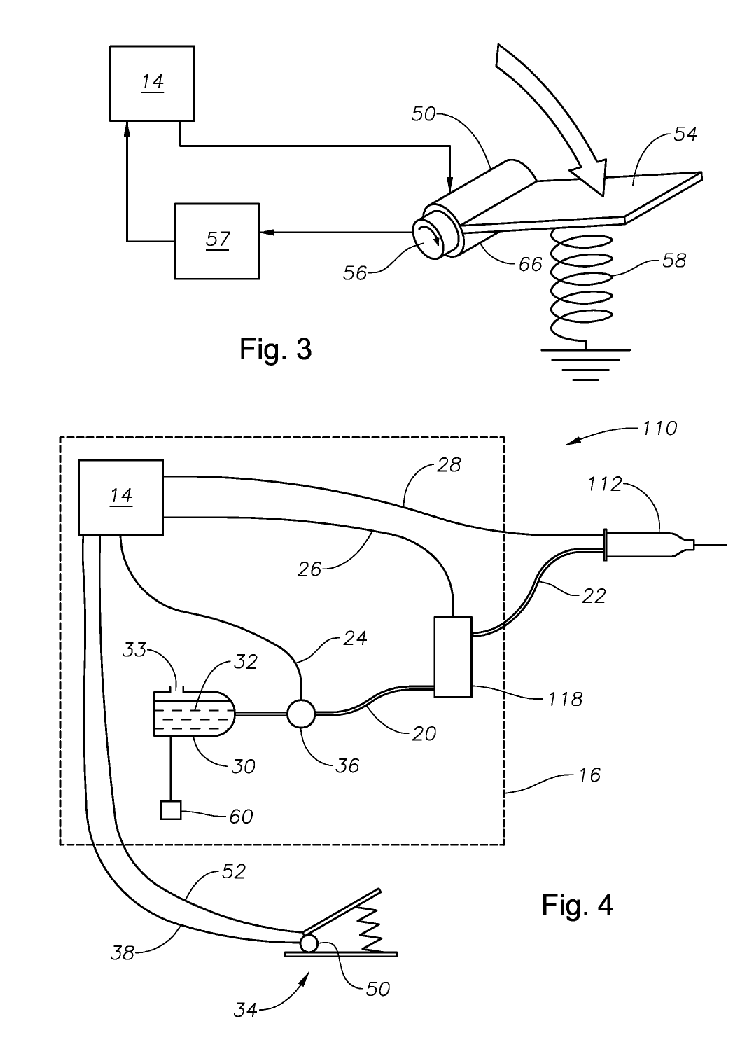

System 10 also includes proportional controller 34. Proportional controller 34 is preferably a foot-pedal type controller, but may be any type of proportional controller appropriate for microsurgery. As best shown in FIG. 3, proportional controller 34 preferably also includes a force feedback motor 50 and an encoder 56. Motor 50 is mechanically coupled to shaft 66 via a conventional gear assembly (not shown). Motor 50 is driven by a signal generated by system 10. Encoder 56 is preferably an optical encoder. Encoder 56 monitors the number of rotations of the shaft of motor 50. Encoder 56 includes position detect logic 57 capable of transforming the number of rotations of shaft of motor 50 into the rotational displacement of pivotable treadle 54. One or more return springs 58 are also coupled to shaft 66. Springs 66 and motor 50 combine to provide a torque or force that resists actuation of treadle 54 by a surgeon's foot. Proportional controller 34 is electrically coupled to computer 14 via interface 38.

During operation, fluid 32, if necessary, is added to reservoir 30 via port 33, and all compressible gas is purged allowing fluid 32 to completely fill tubes 20 and 22 as well as cylinder 42. Reservoir 30 is then pressurized to a predetermined amount. Pressure transducer 36 reads the pressure in tube 20 and transmits this information to computer 14 via interface 24. When the surgeon actuates controller 34 with his or her foot, an electrical signal with a magnitude proportional to the position of treadle 54 is transmitted to computer 14 via interface 38. Computer 14 then supplies a proportional electrical signal to valve 18 via interface 26. This causes valve 18 to begin to open. Because of the proportional nature of system 10, if the surgeon presses treadle 54 closer to the base of controller 34, valve 18 opens further. As valve 18 is opened, pressure is transmitted through tube 22 to cylinder 42. The pressure then acts on actuator 40 causing it to move and actuate the cutting or gripping member of instrument 12. Position of the cutting or gripping member of instrument 12 is transmitted to computer 14 via interface 28 using a conventional position sensor disposed in instrument 12.

Motor 50 functions to provide resistance to treadle 54 of controller 34. If greater force is needed to move the cutting or gripping member of instrument 12 through its complete cycle, such as when attempting to move scissors through thicker or more resistive tissue, computer 14 detects that the cutting or gripping member of instrument 12 has not moved through the complete cycle and signals motor 50 via interface 52 to provide increased resistance to treadle 54. This results in controller 34 having a stiffer feeling to the surgeon when instrument 12 is working in more resistive tissue, thereby allowing system 10 the capability of providing tactile feedback to the surgeon regarding the amount of pressure required to fully actuate instrument 12. Such tactile feedback is not possible with an instrument 12 which is pneumatically actuated due to the compressing of the working gas.

In a second embodiment, best illustrated in FIG. 4, instrument 112 is a vitreous cutter of similar construction to surgical instrument 12. Valve 118 is a simple on/off solenoid valve which is biased in the closed position. During operation, when the surgeon actuates controller 34, an electrical signal is again sent to computer via interface 38. Computer 14 then sends an alternating electrical signal to valve 18 via interface 26, proportional in frequency to the position of treadle 54 of controller 34. The alternating signal causes valve 118 to open and close in rapid succession delivering rapid pulses of pressure to instrument 112. In this embodiment, when control surface 54 is depressed further, the open/close rate of valve 118 is increased, and the cycle rate of instrument 112 increases.

The present invention is illustrated herein by example, and various modifications may be made by a person of ordinary skill in the art. For example, although the microsurgical instruments of the present invention have been described above as having a spring to deliver a restoring force to the actuator, the microsurgical instrument can also be operated with a dual hydraulic drive mechanism having a second tube fluidly coupling reservoir 30 with an opposing side of actuator 40, and a second solenoid valve fluidly coupled to the second tube between reservoir 30 and actuator 40 and electrically coupled to computer 14. In this system, pressure is transmitted to alternating sides of actuator 40, resulting in reciprocal motion. As another example, hydraulic actuator 40 may comprise a linear electric actuator that drives a master diaphragm, bellows, piston, or bourdon actuator disposed in surgical console 16 that is fluidly coupled to slave diaphragm, bellows, piston, or bourdon actuator disposed in instrument 12.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims.

What is claimed is:

1. A microsurgical system comprising:
a microsurgical instrument having a hydraulic actuator disposed therein;
a computer;
a storage reservoir containing a non-compressible hydraulic fluid, said fluid capable of transmitting a force to said actuator;
a tube fluidly coupling said reservoir and said instrument; and
a solenoid valve fluidly coupled to said tube between said reservoir and said instrument and electrically coupled to said computer.

2. The system of claim 1 wherein said instrument further comprises a mechanism to apply a restoring force to said actuator.

3. The system of claim 2 wherein said mechanism is a spring.

4. The system of claim 1 wherein said mechanism comprises:
a second tube fluidly coupling said reservoir with an opposing side of said actuator; and
a second solenoid valve fluidly coupled to said second tube between said reservoir and said instrument and electrically coupled to said computer.

5. The system of claim 1 wherein said instrument is a vitreous cutter.

6. The system of claim 1 wherein said instrument is powered scissors.

7. The system of claim 1 wherein said instrument is powered forceps.

8. The system of claim 1 further comprising a proportional controller having a pivotable treadle, and a motor electrically coupled to said computer, said motor capable of delivering active variable resistance to said treadle.

9. The system of claim 8 wherein resistance provided by said motor is determined by comparing an amount of pressure applied to said actuator to a position of said actuator.

10. The system of claim 9 wherein said motor delivers more resistance to said treadle when said instrument is operating on more resistive tissue.

11. The system of claim 1 wherein said fluid is saline.

12. The system of claim 1 wherein said fluid is deionized water.

13. A method of powering a microsurgical instrument comprising the steps of:
providing a microsurgical system comprising:
a microsurgical instrument having a hydraulic actuator disposed therein;
a computer;
a storage reservoir containing a non-compressible hydraulic fluid;
a tube fluidly coupling said reservoir and said instrument;
a valve fluidly coupled to said tube between said reservoir and said instrument and electrically coupled to said computer; and
a pressure source;
purging said reservoir of any compressible gas;
pressurizing said reservoir with said pressure source; and
opening and closing said valve in response to a signal from said computer to move said actuator with said hydraulic fluid.

14. The method of claim 13 wherein said valve is a proportional solenoid valve.

15. The method of claim 13 wherein said microsurgical system further comprises a proportional controller have a pivotable treadle, and a motor electrically coupled to said computer, and further comprising the step of delivering active variable resistance to said treadle with said motor.

16. The method of claim 15 wherein resistance provided by said motor is determined by comparing an amount of pressure applied to said actuator to a position of said actuator.

17. The method of claim 16 wherein said motor delivers more resistance to said treadle when said instrument is operating on more resistive tissue.

18. The method of claim 13 wherein said instrument is a vitreous cutter.

19. The method of claim 13 wherein said instrument is powered scissors.

20. The method of claim 13 wherein said instrument is powered forceps.

21. The method of claim 14 wherein said fluid is saline.

22. The method of claim 14 wherein said fluid is deionized water.
